(19) United States
(12) Patent Application Publication
(10) Pub. No.: US 2018/0214125 A1
(43) Pub. Date: Aug. 2, 2018

(54) Ultrasound Gel Module Assemblies

(71) Applicant: BURL Concepts, Inc., San Diego, CA (US)

(72) Inventors: Jeffry Tola, Canterbury, CT (US); Thilo Hoelscher, San Diego, CA (US); Thomas Francis Doyle, Encinitas, CA (US)

(73) Assignee: BURL Concepts, Inc.

(21) Appl. No.: 15/883,880

(22) Filed: Jan. 30, 2018

Related U.S. Application Data
(60) Provisional application No. 62/452,253, filed on Jan. 30, 2017.

(51) Int. Cl. A61B 8/00

(52) U.S. Cl. CPC A61B 8/4281; A61B 8/4444

(57) ABSTRACT

Systems and methods for fabricating and using ultrasound gel module assemblies in accordance with embodiments of the invention are illustrated. One embodiment includes an ultrasound gel module assembly including a gel pad attached to a base component, where the gel pad is capable of conducting ultrasound, and a cover component covering a first side of the gel pad and in contact with the base component. In another embodiment, a method for using an ultrasound gel module assembly includes removing a cover component from a gel module assembly, where the cover component covers a first side of a gel pad attached to a base component, and the base component includes a set of attachment points configured to enable the base component to be connected to a holder, attaching the base component to the holder, and transmitting ultrasound through the gel pad from an ultrasound transducer connected to the holder.

### CROSS REFERENCE TO RELATED APPLICATIONS

This application claims priority to U.S. Provisional Application No. 62/452,253 filed Jan. 30, 2017. The disclosure of U.S. Provisional Application No. 62/452,253 is fully incorporated herein by reference.

### FIELD OF THE INVENTION

The present invention relates generally to ultrasound wave transmission, and more specifically to apparatuses and methods for manufacturing ultrasound gel modules.

### BACKGROUND

Ultrasound waves are often described as sound waves having frequencies greater than 20 kHz. Ultrasound has been used in the medical field to observe the interior of the human body in a non-invasive manner. The ultrasound is applied using a transducer that typically comes into contact with the patient's skin. Ultrasound is readily absorbed in air, so gel is often used between the transducer and the skin to enhance the transmission of ultrasound. In some cases, the gel is a liquid substance. In other cases, a gel pad is used where the ultrasound gel is molded into semi-solid disks.

A gel is a solid jelly-like material that can have properties ranging from soft and weak to hard and tough. Gels are defined as a substantially dilute cross-linked system, which exhibits no flow when in the steady-state. By weight, gels are mostly liquid, yet they behave like solids due to a three-dimensional cross-linked network within the liquid. It is the crosslinking within the fluid that gives a gel its structure (hardness) and contributes to the adhesive stick (tack). In this way gels are a dispersion of molecules of a liquid within a solid in which the solid is the continuous phase and the liquid is the discontinuous phase.

### SUMMARY OF THE INVENTION

Systems and methods for fabricating and using ultrasound gel module assemblies in accordance with embodiments of the invention are illustrated. One embodiment includes an ultrasound gel module assembly including a gel pad attached to a base component, where the gel pad is capable of conducting ultrasound, and a cover component covering a first side of the gel pad and in contact with the base component.

In another embodiment, the base component includes a set of cavities, and the gel pad is attached to the base component by filing the cavities.

In a further embodiment, the base component is circular, and set of cavities includes 12 cavities evenly spaced 30 degrees apart on an inner surface of the base component.

In still another embodiment, the base component includes a flange which retains the gel pad via friction on an inner surface of the flange, and the flange anchors the cover component via friction on an outer surface of the flange.

In a still further embodiment, the base component includes at least one structure for retaining the gel pad selected from the group consisting of a blind hole, a through-hole, a spike, and a bump.

In yet another embodiment, the base component includes a set of attachment points configured to enable the base component to be connected to a holder.

In a yet further embodiment, the set of attachment points includes 4 attachment points symmetrically spaced 90 degrees apart on the base component.

In another additional embodiment, the base component includes at least one structure that deforms the gel pad to modify acoustic lensing properties of the gel pad.

In a further additional embodiment, the cover component is removable to expose the first side of the gel pad.

In another embodiment again, the cover component includes a tab to assist in the removal of the cover component from the base component.

In a further embodiment again, the ultrasound gel module assembly further includes a seal covering a second side of the gel pad and adhered to the base component, wherein the seal is removable to expose the second side of the gel pad.

In still yet another embodiment, the seal includes a sheet of aluminum.

In a still yet further embodiment, the ultrasound gel module assembly is capable of attaching to an ultrasound transducer and conducting ultrasound for use in ultrasound imaging.

In still another additional embodiment, the impedance of the gel pad is within 2% of the impedance of water.

In a still further additional embodiment, the ultrasound gel module assembly is stored in a hygienic package.

In still another embodiment again, the hygienic package is a sealed plastic bag.

In a still further embodiment again, at least one side of the gel pad has a geometry that focuses the shape of an ultrasound field passing through the gel pad.

In yet another additional embodiment, the first side of the gel pad and the second side of the gel pad have geometries that focus the shape of an ultrasound field passing through the gel pad, and the geometries of the first side of the gel pad and second side of the gel pad are different geometries.

In a yet further additional embodiment, an ultrasound gel module assembly includes a gel pad attached to a base component, where the gel pad is capable of conducting ultrasound, a cover component covering a first side of the gel pad and in contact with the base component, where the cover component includes a tab to assist with removing the cover component, and a seal covering a second side of the gel pad and adhered to the base component, wherein the base component is a ring structure comprising a first surface and a second surface, where the first surface includes a structure for retaining the gel pad, and the second surface comprises a set of attachment points.

In yet another embodiment again, a method for using an ultrasound gel module assembly includes removing a cover component from a gel module assembly, where the cover component covers a first side of a gel pad attached to a base component, and the base component includes a set of attachment points configured to enable the base component to be connected to a holder, attaching the base component to the holder using the attachment points, and transmitting ultrasound through the gel pad from an ultrasound transducer connected to the holder.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

FIG. 1 conceptually illustrates use of an ultrasound gel module assembly to couple ultrasound generated by an ultrasound transducer to a patient during a procedure involving application of ultrasound using an ultrasound gel module assembly in accordance with an embodiment of the invention.

FIGS. 2A and 2B provide views of an ultrasound gel module assembly in accordance with an embodiment of the invention.

FIG. 3A is a top down view of an ultrasound gel module assembly in accordance with an embodiment of the invention.

FIG. 3B is a cross-sectional view of an ultrasound gel module assembly in accordance with an embodiment of the invention.

FIG. 4A is a bottom view of a base component of an ultrasound gel module assembly in accordance with an embodiment of the invention.

FIG. 4B is a cross-sectional view of a base component of an ultrasound gel module in accordance with an embodiment of the invention.

FIGS. 4C and 4D are views of a base component of an ultrasound gel module in accordance with an embodiment of the invention.

FIGS. 5A, 5B, and 5C provide views of an ultrasound gel module assembly in a transducer holder in accordance with an embodiment of the invention.

FIGS. 6A, 6B, and 6C provide top, bottom, and views of a base sheet in accordance with an embodiment of the invention.

FIGS. 6D, 6E, and 6F provide top, bottom, and views of a cover sheet in accordance with an embodiment of the invention.

FIGS. 7A, 7B, and 7C provide views of a base-cover assembly in accordance with an embodiment of the invention.

FIG. 8 provides a view of a base-cover assembly filled with ultrasound gel in accordance with an embodiment of the invention.

FIGS. 9A and 9B provide top and bottom views of a base-cover assembly with ultrasound gel module assembly extracted in accordance with an embodiment of the invention.

FIGS. 9C and 9D provide top and bottom views of an ultrasound gel module assembly after extraction from a base-cover assembly in accordance with an embodiment of the invention.

FIG. 10 is a flow chart illustrating a method of manufacturing an ultrasound gel module assembly in accordance with an embodiment of the invention.

FIG. 11 is a flow chart illustrating a method of using an ultrasound gel module assembly in accordance with an embodiment of the invention.

FIGS. 12A, 12B, 12C, 12D, 12E, and 12F provide views of a stepped acoustic lens configuration of an ultrasound gel module assembly with a stepped acoustic lens structure in accordance with an embodiment of the invention.

FIGS. 13A, 13B, 13C, 13D, 13E, and 13F provide views of a stepped acoustic lens configuration of an ultrasound gel module assembly with a slotted acoustic lens structure in accordance with an embodiment of the invention.

FIGS. 14A, 14B, 14C, 14D, 14E, and 14F provide views of a stepped acoustic lens configuration of an ultrasound gel module assembly with an aperture acoustic lens structure in accordance with an embodiment of the invention.

FIGS. 15A, 15B, 15C, 15D, 15E, and 15F provide views of a multilayer acoustic lens configuration of an ultrasound gel module assembly with a stepped acoustic lens structure in accordance with an embodiment of the invention.

FIGS. 16A, 16B, 16C, 16D, 16E, and 16F provide views of an ultrasound gel module assembly with stand-off structures in accordance with an embodiment of the invention.

### DETAILED DESCRIPTION

Turning now to the drawings, ultrasound gel module assemblies, and methods of manufacturing and using ultrasound gel module assemblies are illustrated.

Ultrasound has a wide array of applications across a variety of fields. In the medical field, ultrasound is used to sense the interior of a patient's body in a non-invasive manner. Ultrasound is performed on a patient by bringing an ultrasound transducer in contact with the patient's skin, using said transducer to pulse ultrasound waves into the body, and detecting the reflection of the ultrasound waves. However, ultrasound is readily absorbed in air, and therefore, in order to achieve high fidelity imaging, a gel is often used between the skin and the transducer to remove any pockets of air. In some cases, the gel is semi-solid and formed into disks, but still retains the ability to readily transmit ultrasound.

When in the field, it is difficult for Emergency Medical Technicians (EMTs) to quickly diagnose strokes and other types of brain disorders. Ultrasound gel module assemblies can be used to enhance the speed and quality of field ultrasounds. In many embodiments, ultrasound gel module assemblies have attachment points which can be used to quickly attach the ultrasound gel module assembly to an ultrasound transducer (often via an attachment mechanism). In numerous embodiments, ultrasound gel module assemblies have cover components which keep the gel in a clean or sanitary state prior to usage. Cover components can be quickly and easily removed by a user without displacing or damaging the ultrasound gel or the ultrasound transducer. While much of the description that follows utilizes the example of gel module assemblies developed for use in stroke detection, gel module assemblies in accordance with different embodiments of the invention are not limited to this application and can be utilized in any of a variety of ultrasound applications as appropriate to their specific requirements.

Ultrasound gel module assemblies can be constructed out of components, including, but not limited to, a base component, an ultrasound gel, a cover component, and a seal. In a plurality of embodiments, the base component is constructed in such a way that it can retain the ultrasound gel with minimal chance of the ultrasound gel slipping out of the base component. Base components can have a variety of structures to achieve this, including, but not limited to, blind holes, through-holes, spikes, bumps, and/or any other structure to promote the retention of the ultrasound gel. In numerous embodiments, the base component is embedded in the ultrasound gel.

Base and cover components can be constructed out of solid materials ranging from thermoplastic polymers such as nylon or HDPE, to thermoset polymers such as acrylics or epoxies to metals such as aluminum or brass. However, any number of materials can be used in accordance with the requirements of a given application. In many embodiments, base components and cover components are vacuum formed using thermoplastic sheets. Alternate methods of production include, but are not limited to, injection molding, machining, 3D printing, and casting, or any other production method as appropriate to the requirements of a given application. Base components and cover components can then be fitted together to create monolithic structures which form a mold cavity that defines the size and shape of the gel pad. The cover and base can mechanically coupled using “snap” features, interference press fit, screw threads, among others. The combined base component and cover component can be filled with an ultrasound gel. The opening opposite the cover component can be sealed with a seal. In numerous embodiments, the seal is made of a foil. However, seals can be made of a variety of materials, including, but not limited to, lined paper, lined cardboard, aluminum, or any other material as appropriate to the requirements of a given application. In a plurality of embodiments, once the assembly of the combined base component and cover component with an ultrasound gel is complete and sealed, the assembly can be die cut from the plastic sheets to create a finished ultrasound gel module assembly with a removable seal and cover.

Although the figures and following discussion will provide a detailed description of a number of exemplary embodiments of the ultrasound gel module assembly, it should be understood that any number of different configurations and/or manufacturing processes could be used to achieve the basic goals of the system. For example, any number of shapes and forms could be used in order to better match the surface that ultrasound is being used on. In addition, it should be understood that the figures are merely schematic, and that the relative dimensions of the various elements and their relative spacing are merely exemplary and could be varied by one of ordinary skill in the art while remaining within the bounds of this disclosure.

### Using an Ultrasound Gel Module Assembly

Ultrasound gel module assemblies can be quickly used in the field to perform ultrasounds on patients. By removing the seal of the ultrasound gel module assembly, then attaching to an ultrasound transducer and removing the cover, the exposed ultrasound gel can be used between the patient's skin and the ultrasound transducer to mediate the ultrasound scan. In many embodiments, the ultrasound gel module assembly is secured to an ultrasound transducer using a holder configured to hold both the assembly and the transducer.

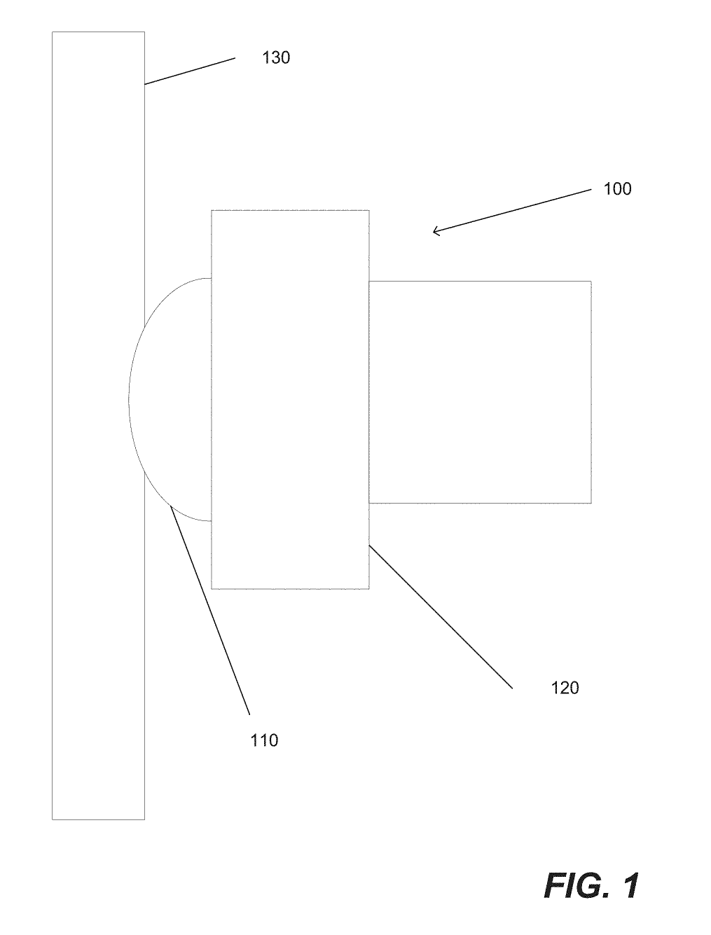

An ultrasound procedure using an ultrasound gel module assembly in accordance with an embodiment of the invention is shown in FIG. 1. The ultrasound gel module assembly 110 can be attached to a transducer 120 to form a usable ultrasound device 100. Once the cover of the ultrasound gel module assembly is removed, the ultrasound device 100 can be placed in contact with the patient's skin 130 such that the ultrasound gel module assembly 110 forms an interface with the patient's skin 130. Sufficient pressure is applied to the patient's skin 130 such that the interface between the ultrasound gel module assembly 110 and the skin 130 has minimal interstitial space. In many embodiments, the transducer 120 is part of a SONAS™ stroke detection system manufactured by BURL Concepts located in San Diego, Calif. In other embodiments, the gel module assembly is utilized with any ultrasound transducer appropriate to the requirements of a given application. In numerous embodiments, the transducer components is embedded into the ultrasound gel module assembly 110, and the ultrasound gel module can be inserted into a housing 120. The form and structure of ultrasound gel module assemblies is discussed below.

### Ultrasound Gel Module Assemblies

Prior to use in an ultrasound procedure, the gel of an ultrasound gel module assembly can be protected by the seal and/or cover components. In numerous embodiments, ultrasound gel module assemblies have their cover components and or seals removed prior to the user performing the ultrasound procedure on the patient. In numerous embodiments, once the seal and cover is removed, the ultrasound gel module assembly effectively transmits ultrasound from the ultrasound transducer to the patient's body.

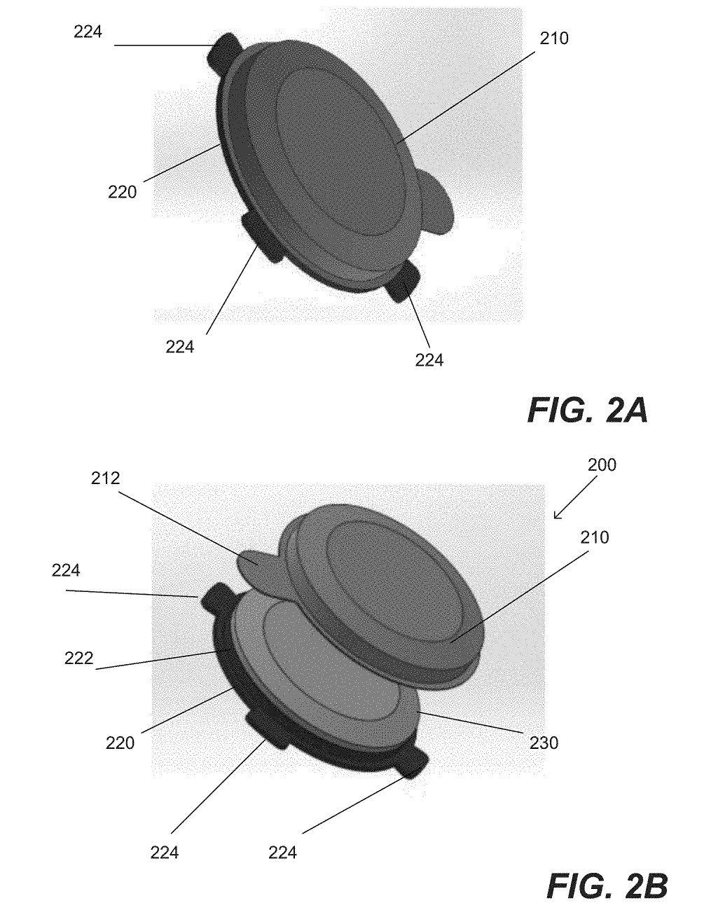

Turning now to FIGS. 2A and 2B, ultrasound gel module assemblies in accordance with an embodiment of the invention are illustrated. In FIG. 2A, an ultrasound gel module assembly with a removable cover in place in accordance with an embodiment of the invention is illustrated. Ultrasound gel module assembly 200 can include cover component 210. In many embodiments, cover component 210 is made of plastic. In a plurality of embodiments, cover component 210 is made of plastic, metal, and/or a metal coated material such as, but not limited to, cardboard and/or plastic. In numerous embodiments, cover component 210 includes one or more tabs 212 which assist in the removal of the cover component. In some embodiments, tab 212 is an extended lip of the cover component 210. Cover component 210 can completely cover the ultrasound gel pad 230 by being in contact with base component 220. In numerous embodiments, cover components are held in place relative to the base component by frictional forces between the cover component and the base component. However, any number of mechanisms including, but not limited to mechanical mechanisms (e.g. threading, hooks, etc.) can be utilized to keep cover components and base components together as appropriate to the requirements of specific applications of embodiments of the invention. In a plurality of embodiments, cover component 210 forms a seal with base component 220. Base component 220 can be made out plastic, metal, or any other material suitable to provide structure in accordance with various embodiments of the invention. In many embodiments, base component 220 has one or more attachment points 224. Attachment points 224 can extend past cover component 210, and can allow the ultrasound gel module assembly 200 to attach to an ultrasound transducer and/or a transducer holder. In some embodiments, base component 220 has four attachment points 224 arranged at 90 degree angles from one another. Attachment points 224 can make up a 22.5 degree arc of a circular base component 220. As can readily be appreciated, the specific attachment points typically depend on the requirements of a given application.

In FIG. 2B, an ultrasound gel module assembly in accordance with an embodiment of the invention is illustrated. Cover component 210 can be removed from base component 220 in order to expose ultrasound gel pad 230. In many embodiments, the base component 220 is in the shape of a ring. In several embodiments, the base component 220 is in any shape appropriate to the requirements of a given application including, but not limited to, the shape of a polygon. Base component 220 can have a flange 222 which can help retain the ultrasound gel pad 230. Flange 222 can provide an anchor for cover component 210.

In many embodiments, ultrasound gel pad 230 is made out of gels such as platinum cured silicones or aqueous based materials like Aquaflex® manufactured by Parker Laboratories, Inc. of Fairfield, N.J. However, any number of ultrasound gels can be used as appropriate to the requirements of a given application. In numerous embodiments, ultrasound gel pad has a composition and geometry such that the acoustic impedance between the transducer and the patient is similar to the resulting impedance if water filled the gap between the transducer and the patient, or an impedance that otherwise is in an acceptable range for the application. Further, with properly matched durometer of the gel pad and design of the base, the ultrasound gel pad can maintain its approximate shape, size, and retention to the base component. In numerous embodiments, ultrasound gel pad 230 has at least a portion of base component 220 embedded inside of the gel. Base component 220 can have one or more protuberances or cavities to assist in retaining the ultrasound gel pad 230. In numerous embodiments, the protuberances are spikes which embed in the ultrasound gel pad 230. In many embodiments, the cavities are through-holes. However, any kind of appropriate protuberance, cavity, or mix of protuberances and cavities can be used as appropriate to the specific gel and/or application in accordance with various embodiments of the invention. In this way, ultrasound gel pad 230 can be attached to base component 220.

Further, in numerous embodiments, base components include structures to modify the acoustic properties of the gel pad. For example, the ultrasound gel module assembly can acquire acoustic lensing properties by changing the structure and form of the base component and/or the gel pad. In numerous embodiments, the base component thickness can be increased or decreased to change the propagation of ultrasound. In a variety of embodiments, structures such as, but not limited to, rings, slats, grids, or any other shape can be utilized to modify acoustic lensing properties of the ultrasound gel module assembly. In many embodiments, materials selection are also varied to modify acoustic impedance. For example, materials of varying acoustic impedance may be applied in layers so that reflective boundaries are formed. In numerous embodiments, when the ultrasound gel is injected during manufacturing to form the gel pad, the structures can change the shape of the pad, further modifying the acoustic properties.

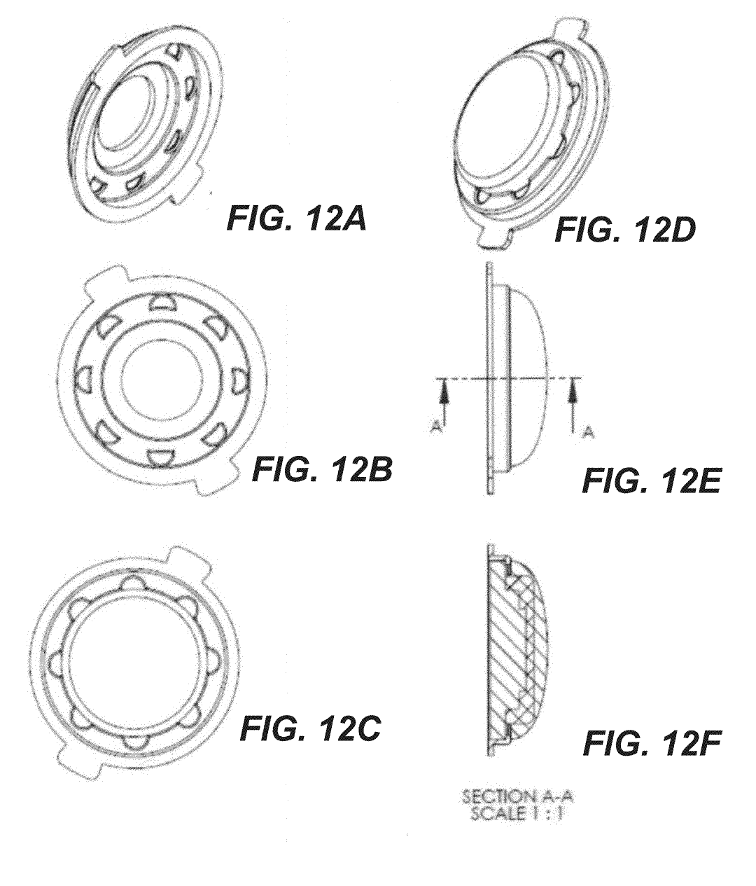

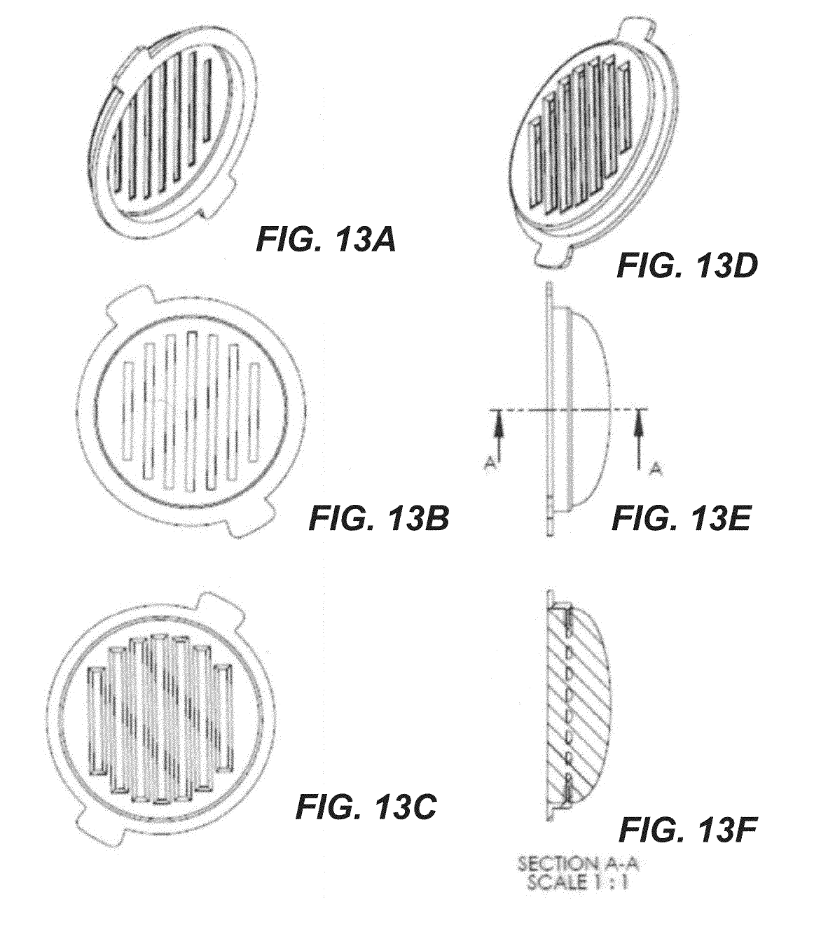

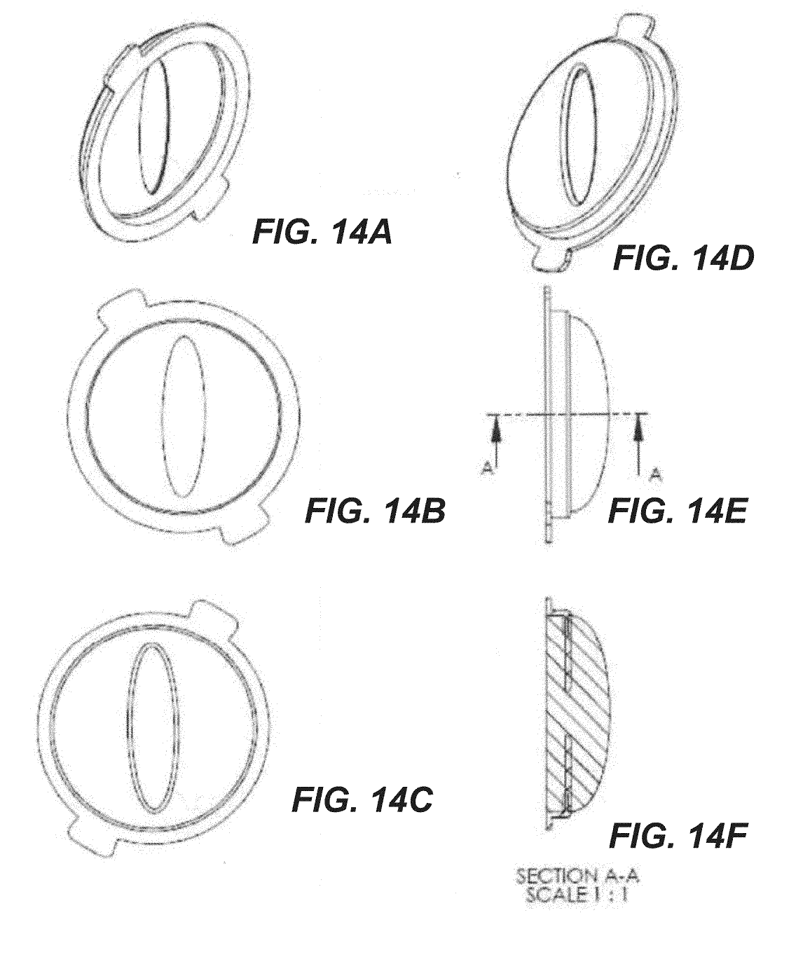

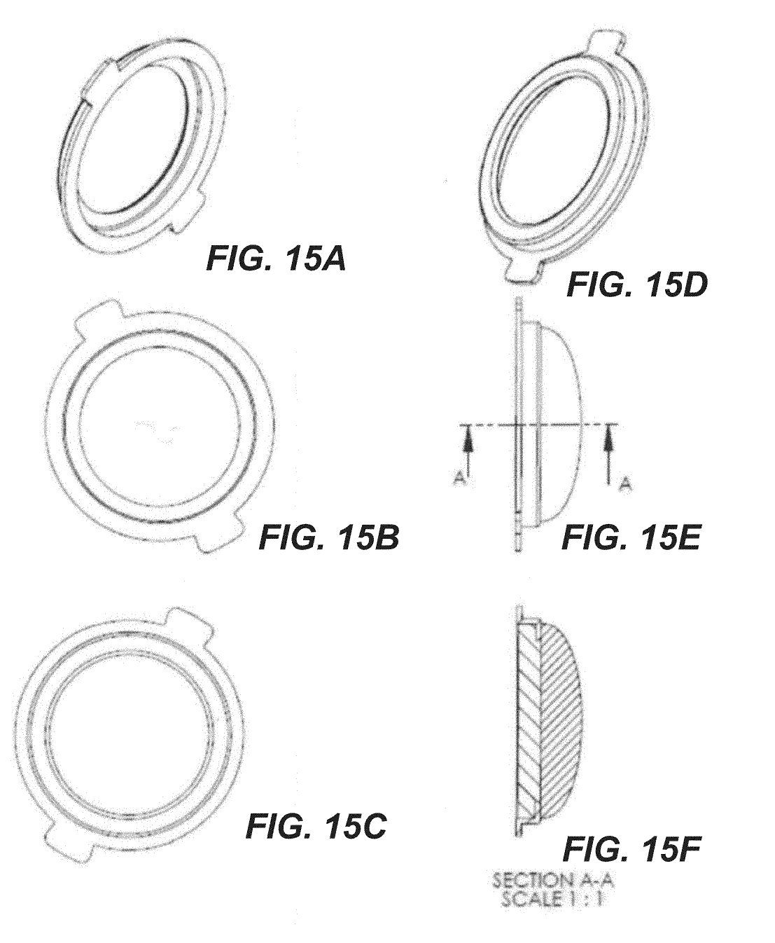

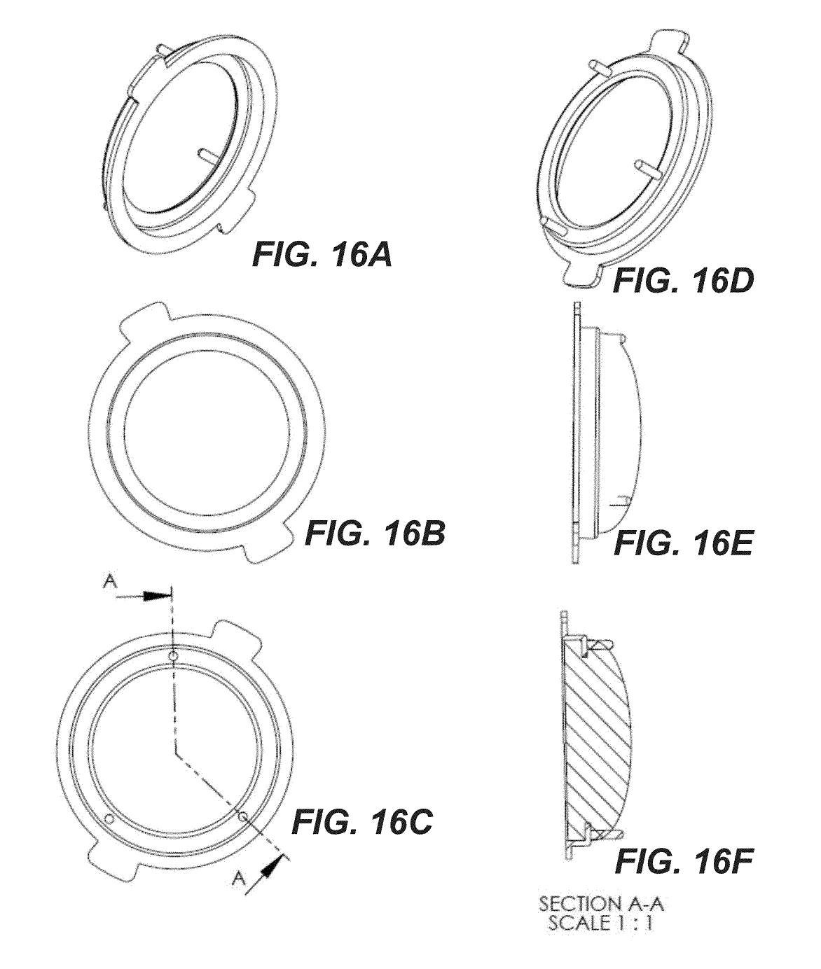

Examples of different types of acoustic lensing structures are illustrated in FIGS. 12A-F, 13A-F, 14A-F, and 15A-F in accordance with embodiments of the invention. Additionally, base components can include structures that enable the ultrasound transducer to be positioned a fixed distance from the patient's skull such as, but not limited to, stand-offs. Such structures can reduce variable readings due to irregular testing surface shapes. An example of an ultrasound gel module assembly with stand-offs is illustrated in FIGS. 16A-F in accordance with an embodiment of the invention. While specific acoustic lensing structures and stand-offs have been illustrated with respect to FIGS. 12A-F, 13A-F, 14A-F, 15A-F, and 16A-F, one of ordinary skill in the art can appreciate that any number of different lensing structures, including structures that utilize different numbers or shapes of apertures, combinations of different aperture types, and/or different numbers or shapes of stand-offs can be used as appropriate to the requirements of specific applications of embodiments of the invention. Ultrasound gel module assemblies can be made from various components. Descriptions of ultrasound gel module assembly components is found below.

### Ultrasound Gel Module Assembly Components

In many embodiments, ultrasound gel module assemblies have base components which retain ultrasound gel modules. The ability to retain ultrasound gel modules within a base component allows EMTs to rapidly connect ultrasound gel modules to ultrasound transducers (either directly or using transducer holders). This increase in speed allows for quick diagnosis and/or triage of patients. In order to retain an ultrasound gel within a base component, a variety of mechanisms can be used in accordance with various embodiments of the invention.

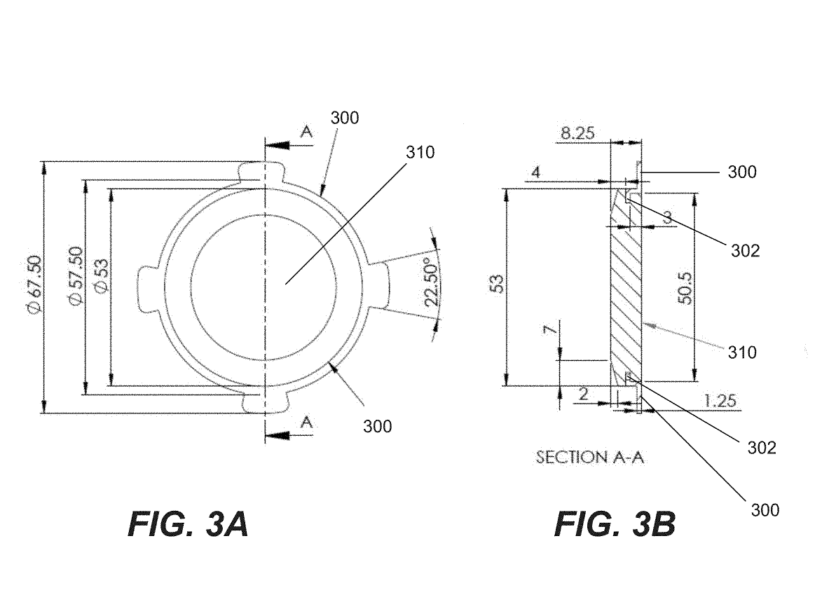

Turning now to FIGS. 3A and B, various views of an ultrasound gel module assembly in accordance with an embodiment of the invention are illustrated. FIG. 3A illustrates a top down view of an ultrasound gel module assembly in accordance with an embodiment of the invention. Base component 300 houses ultrasound gel pad 310. FIG. 3B illustrates a cross-sectional view of the ultrasound gel module assembly with the cover removed. In many embodiments, base component 300 has protuberances 302 which are embedded in ultrasound gel pad 310. In a plurality of embodiments, protuberances 302 are separate protuberances which are spaced across the base component 300. In numerous embodiments, a single protuberance 302 extends from the inner perimeter of the base component 300.

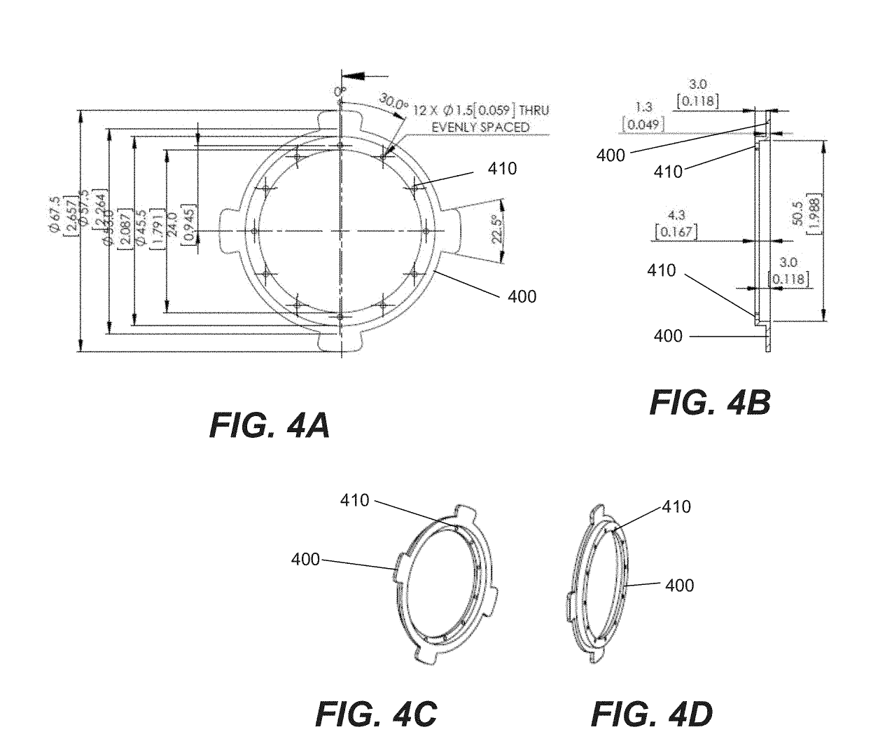

Turning now to FIGS. 4A-D, a base component in accordance with an embodiment of the invention is illustrated. Specifically, FIG. 4A depicts a top view of base component 400. In numerous embodiments, base component 400 has one or more cavities 410. In some embodiments, cavities 410 are through-holes which are holes which pass entirely through base component 400 such that there are two or more openings to the cavity. In many of embodiments, base component 400 has 12 cavities 410 that are evenly spaced 30 degrees apart. FIG. 4B is a cross-sectional view of the base component 400 illustrating that the base component has cavities 410 formed as through-holes in accordance with many embodiments of the invention. FIGS. 4C and 4D show perspective views of base component 400 illustrating the location of the cavities 410. While specific embodiments of the invention are described above with respect to FIGS. 4A-4D, any of a variety of base component structures can be used in the construction of gel module assemblies as appropriate to the requirements of specific applications in accordance with embodiments of the invention. Methods and structures for socketing ultrasound gel module assemblies into transducer holders and/or appropriately configured transducers are described below.

### Socketing Ultrasound Gel Module Assemblies

Ultrasound gel module assemblies can be quickly socketed into transducer holders and/or attached to appropriately configured transducers. In many embodiments, transducer holders are utilized that have a socket for an ultrasound gel module assembly and a socket for an ultrasound transducer. One or more attachment points on base components can be formed that match complementary attachment points on transducer holders, allowing quick and stable connections. Attachment between the ultrasound gel module assembly and the transducer holder can be sufficiently strong to allow the quick removal of the cover component, allowing an EMT to rapidly begin the ultrasound procedure. In certain embodiments, transducer holders are part of a positioning apparatus such as (but not limited to) a positioning headband, or positioning garment.

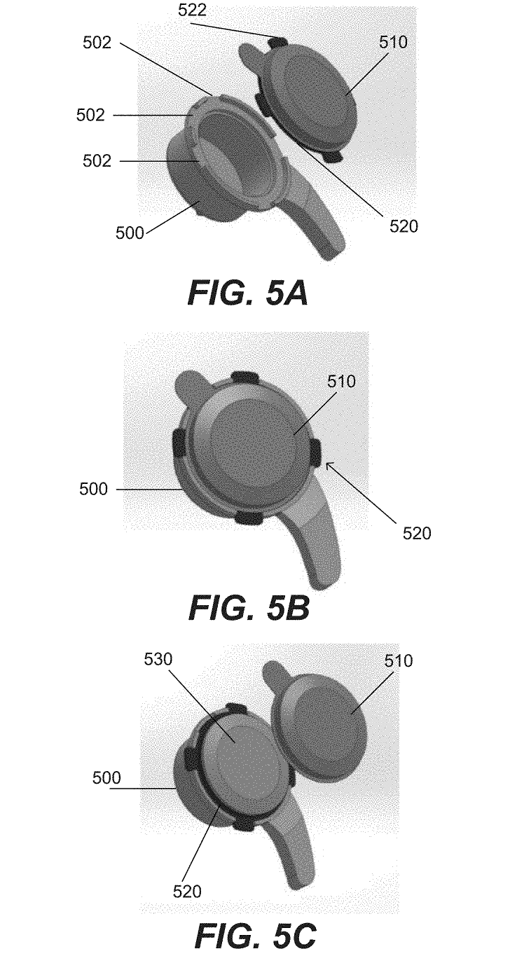

Turning now to FIG. 5A, socketing of the ultrasound gel module into a transducer holder is illustrated in accordance with embodiments of the invention. In many embodiments, transducer holder 500 has one or more complementary attachment points 502. The ultrasound gel module assembly base component 520 can have one or more attachment points 522 which can attach to complementary attachment points 502. In the illustrated embodiment, the complementary attachment points 502 are shown on a transducer holder but could equally be directly incorporated within an ultrasound transducer design, and/or in an adapter piece that can be attached to the transducer housing. Complementary attachment points 502 can be sockets, clasps, or any other mechanism to receive or grasp an ultrasound gel module assembly by the attachment points 522 on the base component 520 in accordance various with embodiments of the invention. In this manner, an ultrasound gel module assembly can be attached to transducer holder 500. In numerous embodiments, cover component 510 is on the ultrasound gel module assembly while the assembly is being inserted into transducer holder 500.

Turning now to FIG. 5B, an embodiment of the ultrasound gel module assembly attached to a transducer holder is illustrated. In many embodiments, transducer holder 500 is connected to ultrasound gel module assembly such that the ultrasound gel module assembly will remain attached to the transducer holder 500 during an ultrasound procedure.

Turning now to FIG. 5C, an embodiment of the ultrasound gel module assembly attached to a transducer holder with cover component removed is illustrated. In many embodiments, cover component 510 is removed from the ultrasound gel module assembly once the ultrasound gel module assembly is attached to transducer holder 500. In numerous embodiments, removing cover component 510 exposes ultrasound gel pad 530. Ultrasound gel pad 530 can be held in place against transducer holder 500 by base component 520. The ultrasound gel pad 530 can be acoustically transparent. In many embodiments, transmission of ultrasound through the ultrasound gel pad 530 is similar to the transmission of ultrasound through water. In numerous embodiments, the acoustic impedance of the ultrasound gel pad is no more than 2% different from the acoustic impedance of water. The ultrasound gel module assembly can be extracted from transducer holder 500. In several embodiments, once the ultrasound procedure has been completed, the ultrasound gel module assembly can be removed from transducer holder 500, allowing transducer holder 500 to be fitted with a new ultrasound gel module assembly and/or a new ultrasound transducer. Although specific ultrasound gel module assemblies and transducer holders are described above with reference to FIGS. 5A-5C, ultrasound gel module assemblies and transducer holders can take on a variety of form factors and constructions as appropriate to the requirements of specific applications in accordance with certain embodiments of the invention. Processes for manufacturing ultrasound gel module assemblies are discussed below.

### Processes for Manufacturing Ultrasound Gel Module Assemblies

Ultrasound gel module assemblies can be manufactured in such a way as to greatly enhance the cohesion between the ultrasound gel and the base component. Further, manufacturing processes in accordance with many embodiments of the invention can be efficient and economical due to the design of the cover component and the base component. In certain embodiments, the cover component is used to shape the ultrasound gel. In many embodiments, the base component is filled with an ultrasound gel in such a way that the ultrasound gel forms around the base component, enabling the base component to retain the ultrasound gel. As can be readily appreciated, the specific components utilized in the construction of gel module assemblies and the manner in which they are manufactured is largely dependent on the requirements of a given application.

### Construction of Base Components

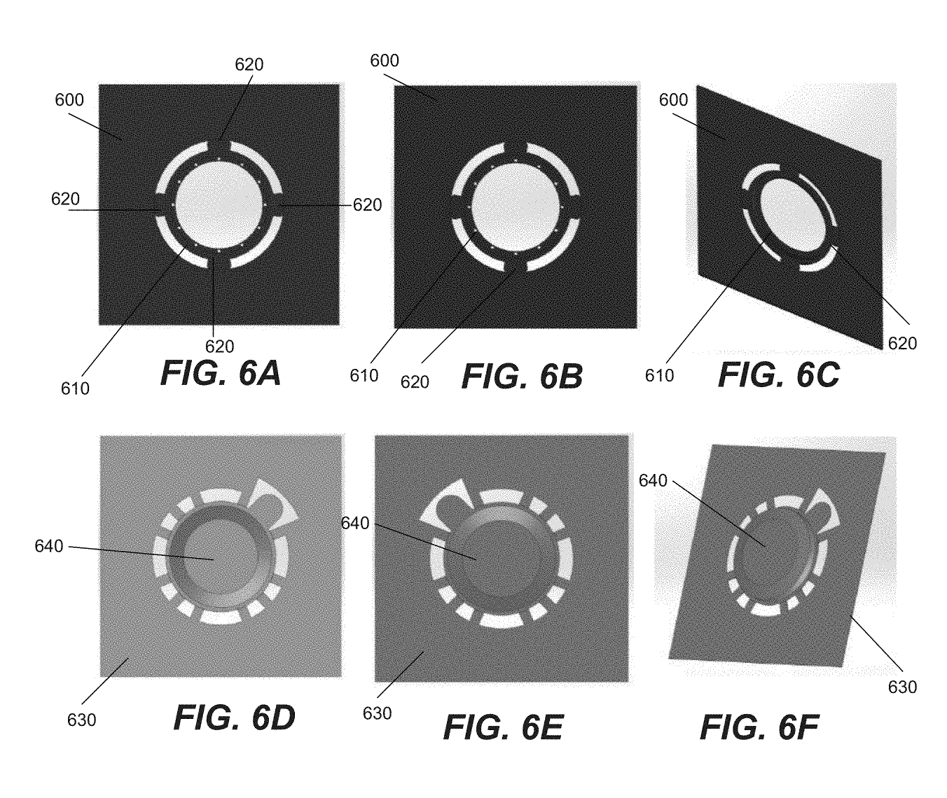

Turning now to FIGS. 6A-C, intermediate steps of a base component manufacturing process are illustrated in accordance with an embodiment of the invention. FIG. 6A illustrates a bottom view of an embodiment of a base component of an ultrasound gel module assembly during manufacture in accordance with an embodiment of the invention. Base sheet 600 can be molded to form a base component 610. Base sheet 600 can be made of a number of different materials, including, but not limited to, metal, plastic, or any other material as appropriate to the requirements of a given application. In numerous embodiments, the material is strong enough to enable robust assembly and retention of the base component to the transducer holder. In a variety of embodiments, the thermal expansion coefficient of the base component material is similar to the transducer holder, the ultrasound transducer, the cover component, and/or the gel pad. In many embodiments, the thermomechanical properties, such as, but not limited to, melting temperature and/or softening temperature are suitable for storage and usage of ultrasound gel module assemblies. In several embodiments, the materials used are capable of being manufactured with a wall thickness between 1 mm and 2 mm with bend radii as low as 0.5 mm. However, materials with other achievable wall thicknesses and bend radii can be used as appropriate to the requirements of a given application.

In numerous embodiments, base sheet 600 can be die cut to form the base component 610 shape. In certain embodiments, base sheet 600 is made using injection. In many embodiments, base sheet 600 is formed using compression molding. In several embodiments, base sheet 600 is formed using thermoforming. In several embodiments, base sheet 600 is formed using stamping techniques, However, one of ordinary skill in the art would appreciate that any number of fabrication techniques, including, but not limited to, 3D printing or machining processes could be used to form sheet 600 into a base component 610 shape. Base component 610 can be held to the remainder of the plastic sheet 600 by one or more attachment points 620. FIG. 6B shows a top view of the base component during manufacture, and FIG. 6C shows a perspective view of the base component.

### Construction of Cover Components

FIG. 6D illustrates a bottom view of the cover component during manufacture. Cover sheet 630 can be molded to form a cover component 640. In numerous embodiments, cover sheet 630 can be die cut to form the cover component 640 shape. In certain embodiments, cover sheet 630 is formed using injection molding techniques. In many embodiments, cover sheet 630 is formed using compression molding. In several embodiments, cover sheet 630 is formed using thermoforming. However, one of ordinary skill in the art would appreciate that any number of fabrication techniques, including, but not limited to, 3D printing or machining processes could be used to form cover sheet 630 into a cover component 640 shape. FIG. 6E shows a top view of the cover component during manufacture, and FIG. 6F shows a schematic view of the cover component.

### Combining Base Sheets and Cover Sheets

Base sheets and cover sheets can be constructed separately and out of different materials. In many embodiments, both sheets can be fit together in a base-cover assembly which forms a mold for the ultrasound gel. In this way, a separate process that removes the gel pad from the mold and then inserts the cover can be eliminated. Further, using the combined sheets as a mold for the ultrasound gel can allow the ultrasound gel to form to the base component, strengthening the cohesion between the ultrasound gel and the base component.

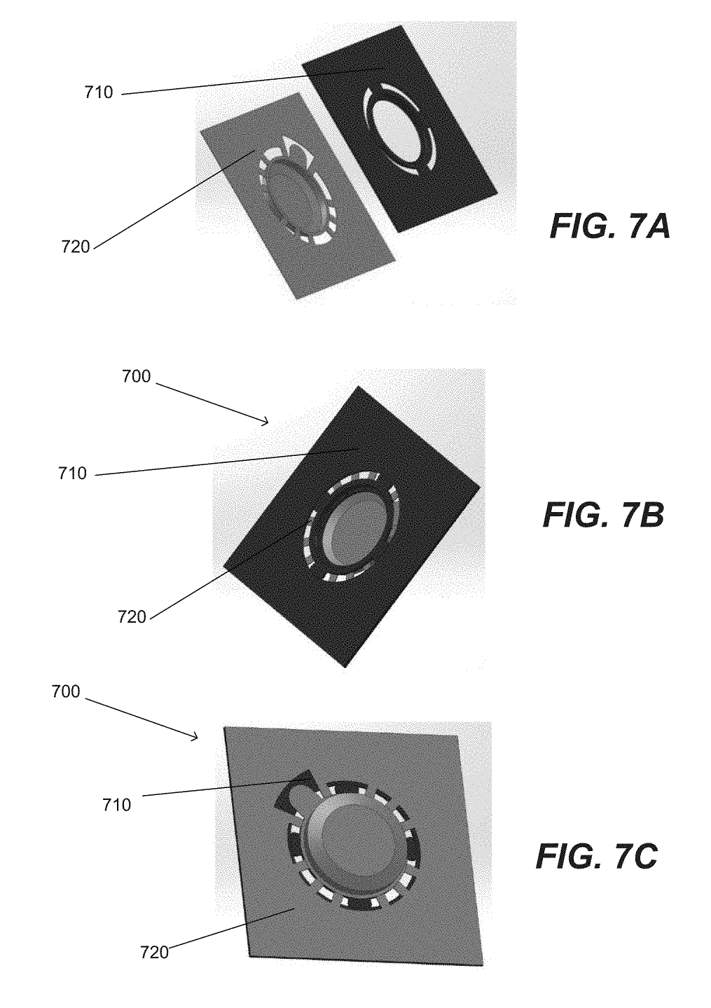

Turning now to FIGS. 7A-C, intermediate steps in combining a base sheet and a cover sheet in accordance with an embodiment of the invention are illustrated. FIG. 7A conceptually illustrates a process involving fitting a base sheet to a cover sheet in accordance with an embodiment of the invention. Base sheet 710 can be placed parallel to cover sheet 720 such that the concave surface of the cover component in the sheet 720 is facing the base sheet 710 such that the flange of base sheet 710 is facing cover sheet 720. While specific processes for joining base and cover components and specific points of attachment are described above with respect to FIG. 7A, the specific process and attachment points are largely dependent upon the requirements of a specific application.

Turning now to FIG. 7B, a perspective view of a base-cover assembly in accordance with an embodiment of the invention is illustrated. In many embodiments, base sheet 710 is attached to cover sheet 720 to form a base-cover assembly 700. Base sheet 710 can be attached to cover sheet 720 through any number of attachment methods, including, but not limited to, an interference fit, a slip-fit, using adhesives, heat stake, or any other attachment method as appropriate to the requirements of specific applications in accordance with various embodiments of the invention. FIG. 7C shows a perspective view of the base-cover assembly.

Molding Ultrasound Gel with Combined Base-Covers

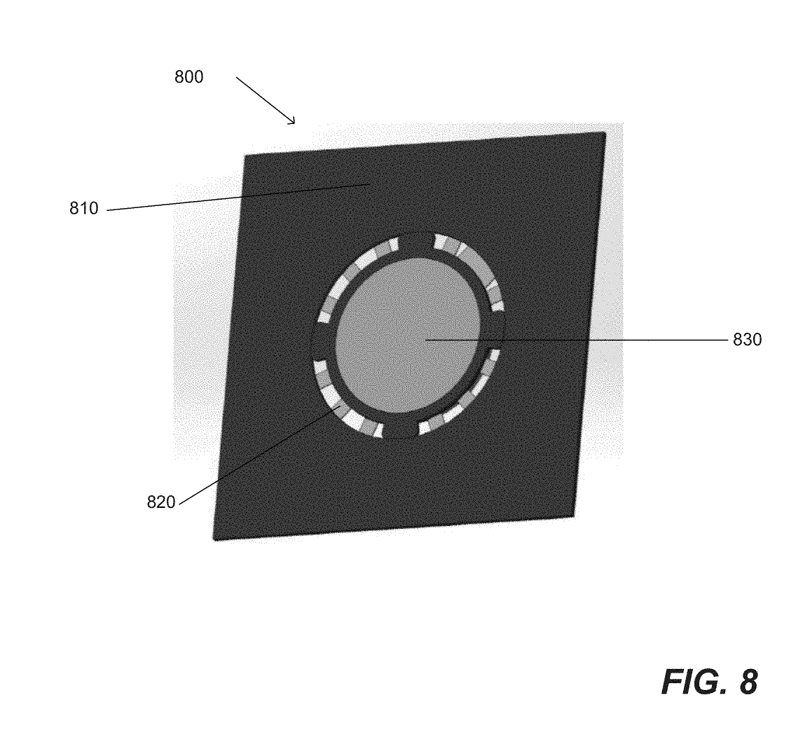

Turning now to FIG. 8, a perspective view of an ultrasound gel filled cover-base assembly is illustrated. The space between base sheet 810 and cover sheet 820 can be filled with an ultrasound gel 830. Ultrasound gel 830 can be introduced into the space between base sheet 810 and cover sheet 820 in such a way that the ultrasound gel 830 is molded by the base sheet 810 and cover sheet 820. In many embodiments, ultrasound gel 830 is poured into the space between base sheet 810 and cover sheet 820 in a more liquid form. The liquid form of the gel can be set into a more solid form in a variety of ways, including, but not limited to, temperature changes, chemical additions, passage of time (drying or curing), or any other method of setting gel in accordance with various embodiments of the invention. In some embodiments, the ultrasound gel 830 liquefies with heat and/or the addition of water. In numerous embodiments, ultrasound gel 830 can be silicon based. In a variety of embodiments, the ultrasound gel 830 is made of Aquaflex® manufactured by Parker Laboratories, Inc. of Fairfield, N.J. However, any number of gel materials can be used as appropriate to the requirements of specific applications in accordance with various embodiments of the invention. In several embodiments, the ultrasound gel 830 is injected into the space between base sheet 810 and cover sheet 820 with sufficient force as to fully fill the space. As can be readily appreciated, the specific gel and technique of introducing the gel into the cavity formed by the cover-base assembly is largely dependent upon the requirements of a given application.

In numerous embodiments, varying amounts of ultrasound gel 830 is introduced into the space between base sheet 810 and cover sheet 820 in order to change the thickness of ultrasound gel 830. By varying the thickness of ultrasound gel 830, the focal distance of the ultrasound gel module assembly can be modified in order to vary the location of the penetration of ultrasound. In several embodiments, an additional molding component is added to the exposed surface of ultrasound gel 830 in order to change the shape of ultrasound gel on the top. The additional molding component can be made of plastic, metal, or any other material as appropriate to the requirements of specific applications in accordance with various embodiments of the invention. In many embodiments, the additional molding component forms the exposed surface of ultrasound gel 830 into a convex shape, however the additional molding component can form the surface of the ultrasound gel 830 into a variety of shapes as appropriate to the requirements of specific applications in accordance with various embodiments of the invention.

In a multitude of embodiments, a first ultrasound gel is molded to a desired shape. The first ultrasound gel can be molded by a cover sheet, by a separate mold, by a combination of a cover sheet and a separate mold, or by any other process as appropriate to the requirements of specific applications in accordance with various embodiments of the invention. A second ultrasound gel can be introduced to the space between base sheet 810 and cover sheet 820. The first ultrasound gel can then be introduced into the space between base sheet 810 and cover sheet 820 such that the first ultrasound gel and the second ultrasound gel are both retained by the base component. In this way, an ultrasound gel module assembly can be created with variable geometries on both faces of the ultrasound gel. Variable geometries can allow for conformance to various surfaces of the body with different shapes, and/or focusing of the shape of the ultrasound beam for superior sensing. In many embodiments, a covering is placed over the ultrasound gel and base component, opposite of the cover component. In many embodiments, the covering is made of a thin sheet of metal. In a variety of embodiments, a wax or metal lined paper is used. Molded ultrasound gel module assemblies can be extracted as individual units. Methods for extracting ultrasound gel module assemblies are described below.

### Extracting Ultrasound Gel Module Assemblies

Once formed, ultrasound gel module assemblies can be extracted from the base-cover assembly. In many embodiments, the extracted base-cover assembly is a finished unit. The process for manufacturing of ultrasound gel module assemblies allows quick extraction of the units from the base-cover assemblies. In numerous embodiments, only one cut is needed to extract ultrasound gel module assemblies. The ease of removal of a completed product enhances the speed of manufacture, as well as lowering the cost of manufacture by increasing efficiency.

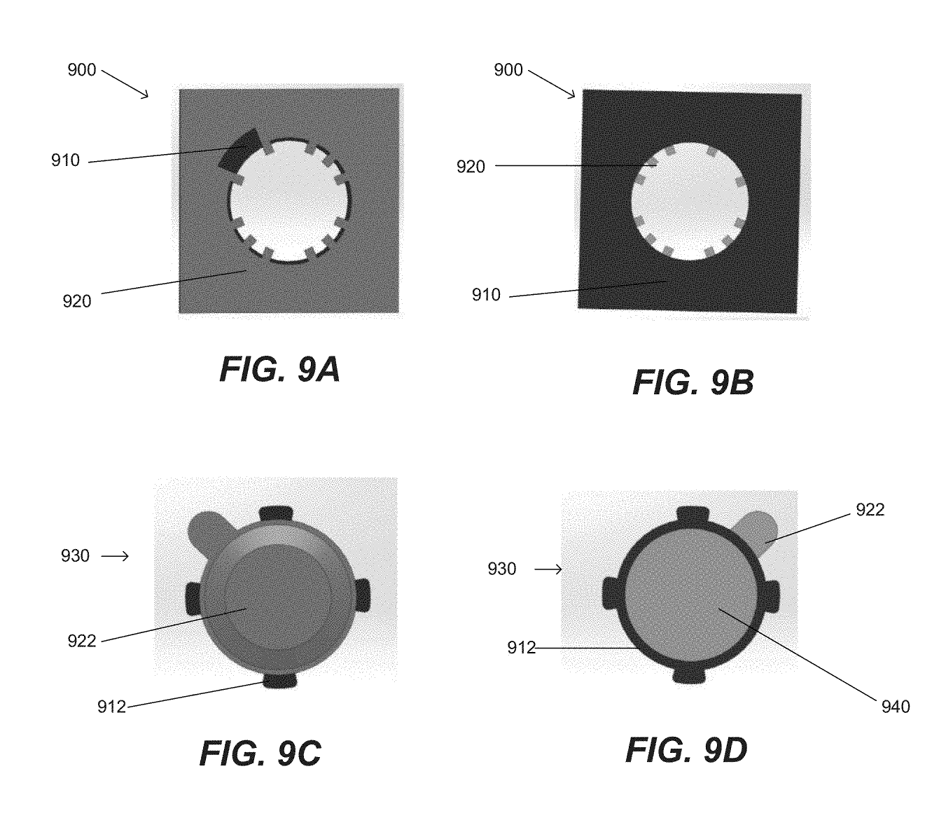

Turning now to FIGS. 9A-D, extraction products are illustrated in accordance with embodiments of the invention. FIG. 9A shows a top view of a base-cover assembly with an ultrasound gel module assembly extracted in accordance with an embodiment of the invention. FIG. 9B shows a bottom view of the base-cover assembly with an ultrasound gel module assembly. Base sheet 910 and cover sheet 920 can remain partially connected. In many embodiments, base sheet 910 and cover sheet 920 are not connected after the ultrasound gel module assembly 930 is extracted. The remaining base sheet 910 and cover sheet 920 can be recycled or reused.

FIG. 9C shows a top view of the ultrasound gel module assembly. FIG. 9D shows a bottom view of the ultrasound gel module assembly. The ultrasound gel module assembly 930 can be extracted from base-cover assembly 900 by die cutting, laser cutting, water jet cutting, or any other cutting mechanism as appropriate to the requirements of specific applications in accordance with different embodiments of the invention. The extraction process separates cover component 922 and base component 912 from the cover sheet 920 and base sheet 910, respectively. Once extracted, ultrasound gel module assembly 930 can be packaged into hygienic packaging. In many embodiments, the hygienic packaging is a blister pack. The blister pack can be made of plastic, metal, or any other material as appropriate to the requirements of specific applications in accordance with various embodiments of the invention. In numerous embodiments, the blister pack is sealed with a thin sheet of aluminum.

### Processes for Manufacturing Ultrasound Gel Module Assemblies

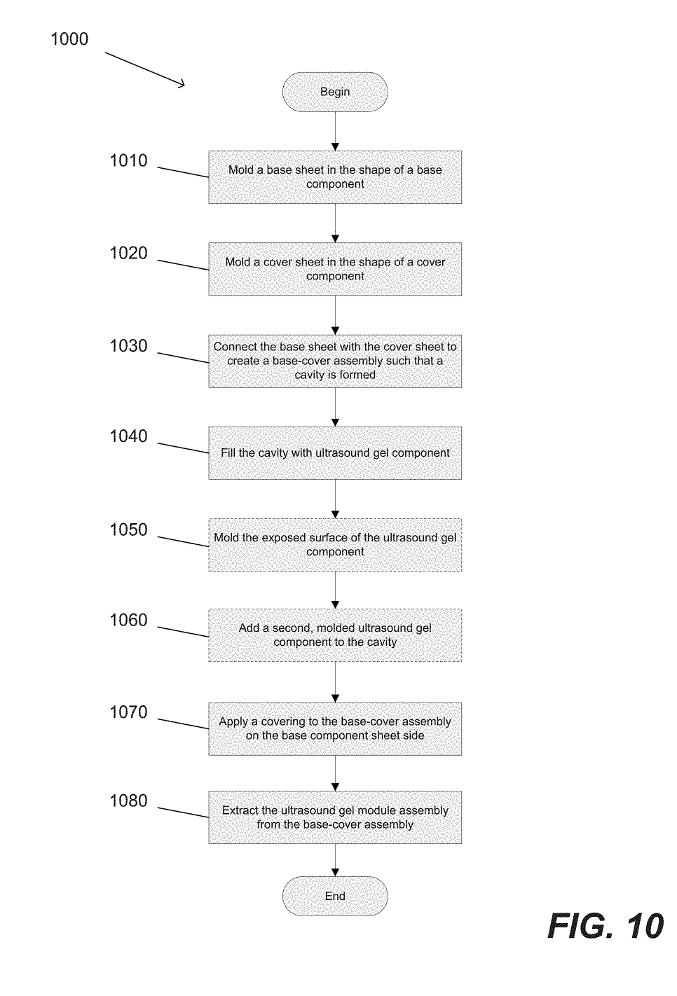

As noted above, ultrasound gel module assemblies can be manufactured in a variety of ways as appropriate to the requirements of specific applications in accordance with embodiments of inventions. Turning now to FIG. 10, a process for manufacturing ultrasound gel module assemblies in accordance with an embodiment of the invention is illustrated. Process 1000 includes molding (1010) a base sheet in the shape of a base component, and molding (1020) a cover sheet in the shape of a cover component. The base sheet and the cover sheet can be connected (1030) to create a base-cover assembly such that a cavity is formed. The cavity can be filled (1040) with ultrasound gel. In many embodiments, the exposed surface of the ultrasound gel is further molded (1050). In a plurality of embodiments, a second molded ultrasound gel is added (1060) to the cavity. A covering can be applied (1070) to the back of the base-cover assembly on the side of the base component. In many embodiments, the covering is a thin sheet of metal. In a variety of embodiments, the covering is made of a wax or metal coated paper product. However, any of a variety of materials can be used for the covering as appropriate to the requirements of a given application. The ultrasound gel module assembly can be extracted (1080). While specific processes are described above with reference to FIG. 10, any of a variety of processes can be utilized for forming an ultrasound gel module assembly as appropriate to the requirements of a given application.

### Processes for Using Ultrasound Gel Module Assemblies

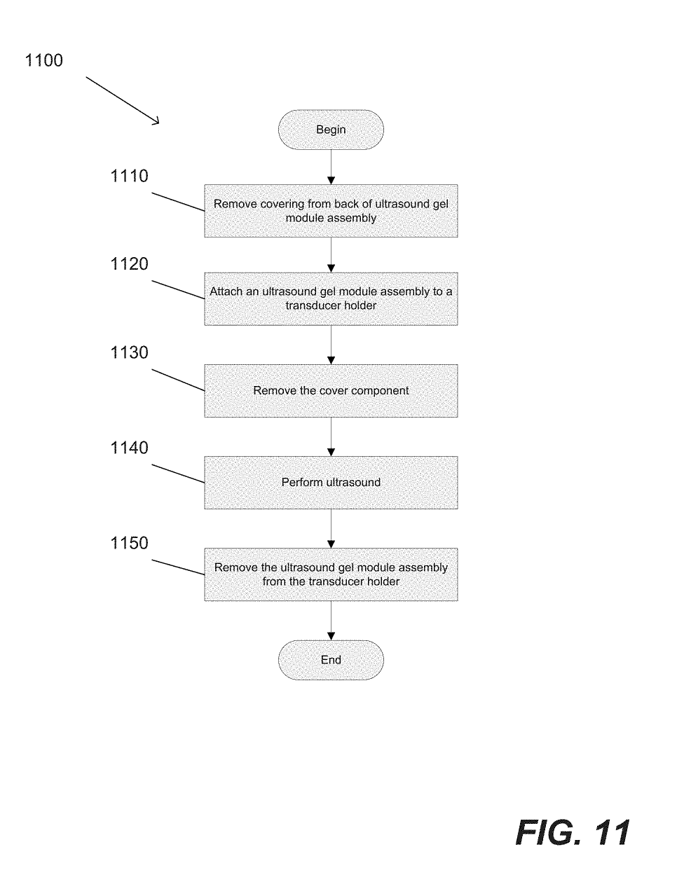

Ultrasound gel module assemblies can be quickly attached to transducer holders, which can allow EMTs or any other user to perform ultrasound procedures rapidly and accurately in the field and/or in a clinical setting. Turning now to FIG. 11, a process for using ultrasound gel module assemblies in accordance with an embodiment of the invention is illustrated. Process 1100 includes removing (1110) the covering from the back of the ultrasound gel module assembly, and attaching (1120) an ultrasound gel module assembly to a transducer holder, and removing (1130) the cover from the ultrasound gel module assembly. The ultrasound can then be performed (1140) on the patient by touching the ultrasound gel module assembly to the patient's skin. In numerous embodiments, additional ultrasound gel or liquid can be applied to the patient's skin and/or the front or back side of the ultrasound gel module assembly to reduce air pockets forming between either the skin and the gel pad or the gel pad and the ultrasound transducer. Once the ultrasound procedure is complete, the ultrasound gel module assembly can be removed (1150) from the transducer holder and disposed of. In some embodiments, the ultrasound gel module assembly can have a cover component reattached, and the ultrasound gel module assembly can be reused. In this way, ultrasound gel module assemblies can be used quickly and safely.

Although the present invention has been described in certain specific aspects, many additional modifications and variations would be apparent to those skilled in the art. In particular, any of the various processes described above can be performed in alternative sequences in order to achieve similar results in a manner that is more appropriate to the requirements of a specific application. It is therefore to be understood that the present invention can be practiced otherwise than specifically described without departing from the scope and spirit of the present invention. Thus, embodiments of the present invention should be considered in all respects as illustrative and not restrictive.

What is claimed is:

1. An ultrasound gel module assembly comprising:
a gel pad attached to a base component, where the gel pad is capable of conducting ultrasound; and
a cover component covering a first side of the gel pad and in contact with the base component.

2. The ultrasound gel module assembly of claim 1, wherein the base component comprises a set of cavities, and the gel pad is attached to the base component by filing the cavities.

3. The ultrasound gel module assembly of claim 2, wherein the base component is circular, and set of cavities comprises 12 cavities evenly spaced 30 degrees apart on an inner surface of the base component.

4. The ultrasound gel module assembly of claim 1, wherein the base component comprises a flange which retains the gel pad via friction on an inner surface of the flange, and the flange anchors the cover component via friction on an outer surface of the flange.

5. The ultrasound gel module assembly of claim 1, wherein the base component comprises at least one structure for retaining the gel pad selected from the group consisting of a blind hole, a through-hole, a spike, and a bump.

6. The ultrasound gel module assembly of claim 1, wherein the base component comprises a set of attachment points configured to enable the base component to be connected to a holder.

7. The ultrasound gel module assembly of claim 1, wherein the set of attachment points comprises 4 attachment points symmetrically spaced 90 degrees apart on the base component.

8. The ultrasound gel module assembly of claim 1, wherein the base component comprises a set of structures that deform the gel pad to modify acoustic lensing properties of the gel pad.

9. The ultrasound gel module assembly of claim 1, wherein the cover component is removable to expose the first side of the gel pad.

10. The ultrasound gel module assembly of claim 9, wherein the cover component comprises a tab to assist in the removal of the cover component from the base component.

11. The ultrasound gel module assembly of claim 1 further comprising a seal covering a second side of the gel pad and adhered to the base component, wherein the seal is removable to expose the second side of the gel pad.

12. The ultrasound gel module assembly of claim 11, wherein the seal comprises a sheet of aluminum.

13. The ultrasound gel module assembly of claim 1, wherein the ultrasound gel module assembly is capable of attaching to an ultrasound transducer and conducting ultrasound for use in ultrasound imaging.

14. The ultrasound gel module assembly of claim 1, wherein the impedance of the gel pad is within 2% of the impedance of water.

15. The ultrasound gel module assembly of claim 1, wherein the ultrasound gel module assembly is stored in a hygienic package.

16. The ultrasound gel module assembly of claim 14, wherein the hygienic package is a sealed plastic bag.

17. The ultrasound gel module assembly of claim 1, wherein at least one side of the gel pad has a geometry that focuses the shape of an ultrasound field passing through the gel pad.

18. The ultrasound gel module assembly of claim 1, wherein the first side of the gel pad and the second side of the gel pad have geometries that focus the shape of an ultrasound field passing through the gel pad, and the geometries of the first side of the gel pad and second side of the gel pad are different geometries.

19. An ultrasound gel module assembly comprising:
a gel pad attached to a base component, where the gel pad is capable of conducting ultrasound;
a cover component covering a first side of the gel pad and in contact with the base component, where the cover component comprises a tab to assist with removing the cover component; and
a seal covering a second side of the gel pad and adhered to the base component;
wherein the base component is a ring structure comprising a first surface and a second surface, where the first surface comprises a structure for retaining the gel pad, and the second surface comprises a set of attachment points.

20. A method for using an ultrasound gel module assembly comprising:
removing a cover component from a gel module assembly, where the cover component covers a first side of a gel pad attached to a base component, and the base component comprises a set of attachment points configured to enable the base component to be connected to a holder;
attaching the base component to the holder using the attachment points; and
transmitting ultrasound through the gel pad from an ultrasound transducer connected to the holder.
